(19) United States
(12) Patent Application Publication
(10) Pub. No.: US 2015/0335542 A1
(43) Pub. Date: Nov. 26, 2015

(54) CONTROLLED DELIVERY WHITENING COMPOSITIONS

(71) Applicant: COLGATE-PALMOLIVE COMPANY, Piscataway, NJ (US)

(72) Inventors: Iraklis PAPPAS, Pennsauken, NJ (US); Shira PILCH, Highland Park, NJ (US); Venda Porter MALONEY, Piscataway, NJ (US); Eric SIMON, Somerset, NJ (US)

(73) Assignee: Colgate-Palmolive Company, Piscataway, NJ (US)

(21) Appl. No.: 14/654,705

(22) PCT Filed: Dec. 21, 2012
(86) PCT No.: PCT/US12/71187

§ 371 Date: Jun. 22, 2015

(51) Int. Cl. A61K 8/24; A61Q 11/00; A61K 8/22

(52) U.S. Cl. CPC A61K 8/24; A61K 8/22; A61Q 11/00; A61K 2800/882; A61K 2800/30

(57) ABSTRACT

Described herein are dual component oral care systems comprising a first component comprising a peroxygen compound and having a first pH and a second component comprising at least one salt of a weak mono or polyprotic acid and having a second pH wherein the second pH is higher than the first pH and is less than 10.0 wherein when combined the first and second components form a tooth-whitening composition having a pH of greater than 6.0 and less than 10.0 is provided.

### BACKGROUND

White teeth are considered to be cosmetically desirable. Oral care formulations comprising various tooth whitening agents are known as being useful for cleaning and whitening teeth. A well-known tooth whitening agent is hydrogen peroxide. The hydrogen peroxide can bleach the teeth, remove stains, and kill cariogenic bacteria. Hydrogen peroxide (H₂O₂) can be used in a variety of oral product forms in order to effect the chemical bleaching/whitening of teeth. One such product form is an oral rinse, or mouthwash.

Formulating H₂O₂ into a stable and effective oral rinse poses a particular challenge given the unstable character of H₂O₂ in aqueous solution. H₂O₂ is known to be unstable with respect to disproportionation. That is, H₂O₂ will spontaneously oxidize to O₂ and reduce to H₂O. Equations 1 and 2 show the reduction potential of H₂O₂ and O₂, respectively. Equation 3 shows the cell potential for the disproportionation of H₂O₂ to H₂O and O₂. The positive value of the cell potential indicates that this reaction is spontaneous. Although thermodynamically spontaneous, the disproportionation of H₂O₂ is known to be highly dependent on solution pH. At lower pH, H₂O₂ is generally stable with respect to disproportionation whilst at higher pH, H₂O₂ is generally unstable with respect to disproportionation. H₂O₂ is stabilized with respect to disproportionation when it is maintained in a low pH environment which is free of materials which may act as catalysts, such as heavy metals.

O₂+2H⁺+2e⁻→H₂O₂ E⁰=0.695V (1)

H₂O₂+2H⁺+2e⁻→2H₂O E⁰=1.776V (2)

2H₂O₂→2H₂O+O₂ E⁰=1.236V (3)

In formulating a whitening oral rinse, it is desirable to have a product which is:
1) Stable on the shelf
2) Effectively and rapidly bleaches teeth during product use

US2005/0249679 describes one strategy for accelerating the whitening of teeth. This document discloses providing a composition comprising a peroxygen compound which is combined prior to use with activating agents including a transition metal catalyst and an alkaline compound. The alkaline compound and transition metal catalyst act synergistically to increase the tooth-whitening activity of the composition and produce more rapid whitening upon application to the teeth.

It would be desirable to provide an oral care composition that has long-term shelf stability but that has enhanced bleaching efficacy during use. Furthermore, it would be desirable to provide an oral care composition that is free of transition metals.

### SUMMARY

The present invention aims to at least partially meet these needs in the art.

According to a first aspect of the present invention there is provided a dual component oral care system comprising
a first component comprising a peroxygen compound and having a first pH and
a second component comprising at least one salt of a weak mono or polyprotic acid and having a second pH
wherein the second pH is higher than the first pH and is less than 10.0
wherein when combined the first and second components form a tooth-whitening composition having a pH of greater than 6.0 and less than 10.0.

Optionally, the second component comprises a salt of pyrophosphoric acid. Further optionally the second component comprises a mixture of two or more salts of pyrophosphoric acid. Further optionally the second component includes tetrasodium pyrophosphate. Further optionally the second component includes disodium pyrophosphate. Still further optionally the second component comprises tetrasodium pyrophosphate and disodium pyrophosphate.

Optionally the second component comprises tetrasodium pyrophosphate and disodium pyrophosphate in a ratio of from 20:1 to 1:20 by weight. Further optionally the second component comprises tetrasodium pyrophosphate and disodium pyrophosphate in a ratio of from 17:1 to 1:17 by weight.

Optionally when combined the first and second components form a tooth-whitening composition having a pH of from 6.0 to 10.0. Optionally when combined the first and second components form a tooth-whitening composition having a pH of from 6.8 to less than 9.0, from 6.8 to 8.5, or from 7.5 to 8.5.

Optionally, when combined the first and second components form a tooth-whitening composition having a pH of about 8.0.

Typically, the first component comprises from 0 to 1% by weight (based on the total weight of the tooth-whitening composition) transition metal groups. Optionally the first component comprises less than 0.1% transition metal groups. Further optionally the first component is substantially free of transition metal groups.

Optionally the pH of the first component is less than or equal to 7.0. Further optionally the pH of the first component is from 1.0 to 7.0, from 4.0 to 7.0, from 4.0 to 6.8, from 4.5 to 5.5 or from 4.8 to 5.2. Typically the pH of the first component is about 5.0.

Optionally the pH of the second component is from 7.1 to less than 9.0. Further optionally the pH of the second component is 7.5 to less than 9.0 or from 7.5 to 8.5.

Typically the pH of the second component is about 8.0.

Optionally the peroxygen compound is selected from one or more of peroxides, perborates, percarbonates, persulfates, perphohosphates, persilicates, peroxyacids, peracetates, and combinations thereof. Optionally the peroxygen compound is a peroxide. Further optionally the peroxygen compound is hydrogen peroxide.

Optionally the peroxygen compound is present in an amount of 0.01 to 10 weight % based on the total weight of the tooth whitening composition. Further optionally the peroxygen compound is present in an amount of 0.01 to 8 weight %, 0.01 to 7 weight %, 0.01 to 5 weight %, 0.01 to 3 weight %, 0.01 to 1% or 0.01 to 0.0% based on the total weight of the tooth whitening composition.

Optionally the peroxygen compound is hydrogen peroxide present in an amount of 0.01 to 5 weight % based on the total weight of the tooth whitening composition.

Optionally the tooth whitening composition is a mouthwash.

Optionally there is provided an oral care system wherein the first pH is acidic, the second pH is alkaline and the pH of the combination of the first and second components is alkaline wherein the second component acts as a buffer and the combination of the first and second components has a pH that is than or equal to the pH of the second component.

According to a further aspect of the invention there is also provided a method of tooth whitening comprising combining

a first component comprising a peroxygen compound and having a first pH and

a second component comprising a salt of a weak mono or polyprotic acid and having a second pH

wherein the second pH is higher than the first pH and is less than 10.0

to form a tooth-whitening composition having a pH of greater than 6.0 and less than 10.0 and applying the tooth-whitening composition to a tooth.

According to a further aspect of the invention there is also provided use of a dual component oral care system as described herein for whitening a tooth.

### DETAILED DESCRIPTION

It should be understood that the detailed description, and specific examples, while indicating embodiments of the invention, are intended for purposes of illustration only and are not intended to limit the scope of the invention. The following description of the preferred embodiments is merely exemplary in nature and is in no way intended to limit the invention, its application, or uses.

As used throughout, ranges are used as shorthand for describing each and every value that is within the range. Any value within the range can be selected as the terminus of the range.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “about”, when applied to the value for a parameter of a composition or method of this invention, indicates that the calculation or the measurement of the value allows some slight imprecision without having a substantial effect on the chemical or physical attributes of the composition or method. If, for some reason, the imprecision provided by “about” is not otherwise understood in the art with this ordinary meaning, then “about” as used herein indicates a possible variation of up to 5% in the value.

As referred to herein, all compositional percentages are by weight of the total composition, unless otherwise specified.

To meet both requirements of a desirable product listed above, it is proposed to provide an oral rinse contained within two compartments or chambers. One chamber contains H₂O₂ (or an alternative peroxygen compound, e.g. peracetic acid) in a low pH environment which is free of materials which may act as catalysts, such as transition metal groups. The second chamber contains a higher pH solution which is combined with the H₂O₂-containing chamber before use by the consumer. Preferably, the second chamber contains a mixture of salts of weak mono or polyprotic acids, which have capacity to buffer the combined chambers at the desired pH. Thus the peroxygen compound is maintained at a low pH, improving stability, until just before use when it is mixed with a buffer of higher pH resulting in a composition of improved whitening efficacy. By increasing the pH of H₂O₂ directly before product use, the bleaching efficacy of H₂O₂ is increased.

It has been found that by using a mixture of salts of weak mono or polyprotic acids, the pH of the final composition can be controlled. The salts of weak mono or polyprotic acids have the capacity to buffer the combination of the first and second components at a desired pH value. In some embodiments, the first and second components when combined are buffered such that the whitening composition has a pH less than or equal to the pH of the second component.

The dual component oral care system of the invention comprises a first component comprising a peroxygen compound and having a first pH, and a second component comprising a salt of a weak mono or polyprotic acid and having a second pH, wherein the second pH is higher than the first pH and wherein the combination of the first and second components is a tooth-whitening composition having a pH of greater than 6.0. Thus the two components are mixed or combined just before use to give a tooth whitening composition of higher pH with improved efficacy.

The peroxygen compound can be any peroxide compound such as a peroxide-based bleaching agent which can deliver a hydrogen peroxide ion or an organic peroxide ion.

The peroxygen compound can be hydrogen peroxide, an organic peroxide compound, a hydrogen peroxide generating compound or combinations thereof. Such organic peroxide compounds can be, for example, urea hydrogen peroxide, glyceryl peroxide, benzoyl peroxide, monoperoxyphthalate or combinations thereof. The hydrogen peroxide generating compound can be, for example, sodium persulfate, sodium dipersulfate, sodium percarbonate, sodium perphosphate, sodium perborate, sodium persilicate, potassium persulfate, potassium dipersulfate, potassium percarbonate, potassium perphosphate, potassium perborate, potassium persilicate, calcium persulfate, calcium dipersulfate, calcium percarbonate, calcium perphosphate, calcium perborate, calcium persilicate, sodium peroxide, potassium peroxide and calcium peroxide or combinations thereof.

Organic peroxide compounds can include, for example, urea hydrogen peroxide (carbamide peroxide), glyceryl hydrogen peroxide, alkyl hydrogen peroxide (R—O—O—H), dialkyl hydrogen peroxide (R—O—O—R′), peroxy acids (RCO—O—O—H), peroxy esters (RCO—OOR′), and diacyl peroxides (R—CO—O—O—CO—R′). Examples of such compounds include diacyl peroxide, benzoyl peroxide and the peroxy acid monoperoxyphthalate.

In some embodiments, the compositions comprise not more than 30% by weight of a peroxygen compound, optionally not more than 8% by weight, optionally not more than 7% by weight, optionally not more than 5% by weight, optionally not more than 3% by weight, optionally not more than 1% by weight. The compositions may comprise from about 0.01 to about 30% by weight of a peroxygen compound, optionally 0.01 to 8% by weight, optionally 0.01 to 7% by weight, optionally 0.01 to 5% by weight, optionally 0.01 to 3% by weight or optionally 0.01 to 1% by weight.

The compositions of the present invention can be applied to a tooth to achieve tooth-whitening. The time period for such application can be referred to as “effective tooth-whitening period”. This represents the time period during which the compositions contact the tooth during a single application. An “effective tooth-whitening period” can be about 10 minutes or less, about 15 minutes or less, about 20 minutes or less, about 25 minutes or less or about 30 minutes or less. Alternatively the “effective tooth-whitening period” can be more than about 30 minutes.

The composition can be applied in a single application or in repeated applications. Such repeated or successive applications can be performed one or more times during the day such as, for example, once a day, twice a day, three times a day. Alternatively successive applications can be less frequent such as, for example, once every 2 days, once every three days or once a week. The application period can continue for, for example, about one week, about 2 weeks, about three weeks or about four weeks or longer.

One or more redox colour indicator that are oxidized by hydrogen peroxide can also be included in the second component of the dual component system. The colour indicator can be a dye suitable for us in a tooth-bleaching composition such as food colour additives certified under the Food Drug & Cosmetic Act for use in food and ingested drugs. For example, dyes including FD&C Red No. 3 (sodium salt of tetriodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-(N-ethyl-N-p-sulfobenzyl)-Δ-3,5-cyclohexadienimine]. FD&C Blue No. 1 (disodium salt of dibenzyldiethyldiaminotriphenylcarbinol trisulfonic acid of indigotin. These dyes can change colour upon contacting peroxide compounds thereby signalling to the user when the effective whitening period is completed. Such dyes (either alone or in combination) can be incorporated into the second component at concentrations of, for example, from about 0.005% to about 0.5% by weight or from about 0.025% to about 0.15% by weight based on the total weight of the composition.

The oral care systems of the present invention may also comprise additional ingredients which are typical to most oral care or mouthrinse composition formulations. The first component or the second component may comprise such additional ingredients or alternatively both the first and second components may comprise additional ingredients.

Among useful carriers for optional inclusion in a system of the invention are diluents, bicarbonate salts, pH modifying agents, surfactants, foam modulators, thickening agents, viscosity modifiers, humectants, sweeteners, flavorants, colorants, anticaries agents, antibacterial agents, desensitizing agents, and anticalculus or tartar control agents. Carriers should be selected for compatibility with each other and with other ingredients of the system.

Water is a preferred diluent and is commonly accompanied by an alcohol, e.g., ethanol. The weight ratio of water to alcohol in a mouthwash composition is generally 1:1 to 20:1, for example 3:1 to 20:1 or 4:1 to 10:1. Thus the tooth whitening composition of the present invention may comprise such a ratio of water to alcohol. In a whitening liquid, the weight ratio of water to alcohol can be within or below the above ranges, for example, 1:10 to 2:1.

In a further embodiment, one or both of the components comprises at least one bicarbonate salt, useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. One or more bicarbonate salts are optionally present in a total amount of about 0.1 wt. % to about 50 wt. %, for example about 1 wt. % to 20 wt. %, by total weight of the composition.

In a still further embodiment, the composition of the invention comprises at least one additional pH modifying agent. Such agents include acidifying agents to lower pH, basifying agents to raise pH, and buffering agents to control pH within a desired range. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and the like.

In a still further embodiment, the composition of the invention comprises at least one surfactant. Any orally acceptable surfactant, most of which are anionic, nonionic or amphoteric, can be used. Suitable anionic surfactants include without limitation, water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation, derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of about 0.01 wt. % to about 10 wt. %, for example, from about 0.05 wt. % to about 5 wt. %, or from about 0.1 wt. % to about 2 wt. % by total weight of the whitening composition.

In a still further embodiment, the composition of the invention comprises at least one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000.000 to 2,500,000. One or more PEGs are optionally present in a total amount of about 0.1 wt. % to about 15 wt. %, for example from about 0.2 wt. % to about 7 wt. %, or from about 0.25 wt. % to about 3 wt. %, by total weight of the composition.

In a still further embodiment, the composition of the invention comprises at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly t-carrageenan (iota-carrageenan), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol® series. Particularly preferred Carbopols include Carbopol 934, 940, 941, 956, 974P, and mixtures thereof. One or more thickening agents are optionally present in a total amount of from about 0.01 wt. % to 15 wt. %, for example from about 0.1 wt. % to about 10 wt. %, or from about 0.2 wt. % to about 5 wt. %, by total weight of the composition.

In a still further embodiment, the composition of the invention comprises at least one viscosity modifier, useful for example to inhibit settling or separation of ingredients or to promote re-dispersibility upon agitation of a liquid composition. Any orally acceptable viscosity modifier can be used, including without limitation, mineral oil, petrolatum, clays and organomodified clays, silica and the like. One or more viscosity modifiers are optionally present in a total amount of from about 0.01 wt. % to about 10 wt. %, for example, from about 0.1 wt. % to about 5 wt. %, by total weight of the composition.

In a still further embodiment, the composition of the invention comprises at least one humectant. Any orally acceptable humectant can be used, including without limitation, polyhydric alcohols such as glycerin, sorbitol, xylitol or low molecular weight PEGs. Most humectants also function as sweeteners. One or more humectants are optionally present in a total amount of from about 1 wt. % to about 70 wt. %, for example, from about 1 wt. % to about 50 wt. %, from about 2 wt. % to about 25 wt. %, or from about 5 wt. % to about 15 wt. %, by total weight of the composition.

In a still further embodiment, a composition of the invention comprises at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount depending strongly on the particular sweetener(s) selected, but typically 0.005 wt. % to 5 wt. %, by total weight of the composition.

In a still further embodiment, a composition of the invention comprises at least one flavorant, useful for example to enhance taste of the composition. Any orally acceptable natural or synthetic flavorant can be used, including without limitation vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, cucalyptol, anethole, eugenol, cassia, oxanone, α-irisone, propenyl guaiethol, thymol, linalool, benzaldehyde, cinnamaldehyde, N-ethyl-p-menthan-3-carboxamine. N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt. % to about 5 wt. %, for example, from about 0.1 wt. % to about 2.5 wt. %, by total weight of the composition.

In a still further embodiment, a composition of the invention may comprise at least one colorant. Colorants herein include pigments, dyes, lakes and agents imparting a particular luster or reflectivity such as pearling agents. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like. One or more colorants are optionally present in a total amount of from about 0.001 wt. % to about 20 wt. %, for example, from about 0.01 wt. % to about 10 wt. %, or from about 0.1 wt. % to about 5 wt. %, by total weight of the composition.

In some embodiments, the composition comprises a fluoride ion source. Fluoride ion sources include, but are not limited to: stannous fluoride, sodium fluoride, potassium fluoride, potassium monofluorophosphate, sodium monofluorophosphate, ammonium monofluorophosphate, sodium fluorosilicate, ammonium fluorosilicate, amine fluoride such as olaflur (N′-octadecyltrimethylendiamine-N,N,N′-tris(2-ethanol)-dihydrofluoride), ammonium fluoride, and combinations thereof. In certain embodiments the fluoride ion source includes stannous fluoride, sodium fluoride, amine fluorides, sodium monofluorophosphate, as well as mixtures thereof. In certain embodiments, the oral care composition of the invention may also contain a source of fluoride ions or fluorine-providing ingredient in amounts sufficient to supply about 50 to about 5000 ppm fluoride ion, e.g., from about 100 to about 1000, from about 200 to about 500, or about 250 ppm fluoride ion. Fluoride ion sources may be added to the compositions of the invention at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts. A preferred fluoride salt may be sodium fluoride.

The composition of the present invention optionally comprises a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

The composition of the present invention optionally incorporates one or more antisensitivity agents, e.g., potassium salts such as potassium nitrate, potassium bicarbonate, potassium chloride, potassium citrate, and potassium oxalate; capsaicin; eugenol; strontium salts; zinc salts; chloride salts and combinations thereof. Such agents may be added in effective amounts, e.g., from about 1 wt. % to about 20 wt. % by weight based on the total weight of the composition, depending on the agent chosen. The compositions of the present invention may also be used to treat hypersensitivity by blocking dentin tubules when applied to a tooth.

In some embodiments, the composition of the invention further comprises an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

In another embodiment, the composition comprises an orally acceptable zinc ion source useful, for example, as an antimicrobial, anticalculus or breath-freshening agent. One or more such sources can be present. Suitable zinc ion sources include without limitation zinc acetate, zinc citrate, zinc gluconate, zinc glycinate, zinc oxide, zinc sulfate, sodium zinc citrate and the like. One or more zinc ion sources are optionally and illustratively present in a total amount of from about 0.05 wt. % to about 3 wt. %, for example from about 0.1 wt. % to about 1 wt. %, by total weight of the composition.

The composition of the present invention may additionally optionally comprise a tartar control (anticalculus) agent as provided below. Tartar control agents among those useful herein include salts of the specified agents, including alkali metal and ammonium salts. The agents include: phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof. Other useful tartar control agents include polycarboxylate polymers and polyvinyl methyl ether/maleic anhydride (PVM/MA) copolymers, such as GANTREZ®.

In some embodiments, the composition of the present invention further comprises a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophan, L-lysine, methionine, threonine, levocarnitine and L-carnitine), lipotropics (such as choline, inositol, betaine, and linoleic acid), and mixtures thereof.

The oral care composition of the present invention preferably comprises an orally acceptable carrier for use in a mouth rinse (including dual phase mouthwash), toothpaste, actives in beads/strips, irrigation fluids, plaque removal fluids, Wisp® formulas, formulations to be delivered through devices such as pens, back of a toothbrush and front of a toothbrush, formulations to be delivered through porous wicking materials, interdental brushes, fluid encased dental strips, floss impregnated or coated with the formulations or dried formulations, portables, oral trays, hard or soft candy, lozenge with a liquid inside, peelable gels, patches, formulations for pop-rocks that upon popping, spread a fine mist of the formulation around oral cavity and dental strips. Accordingly, opportunities exist for professional use of the compositions of the present invention (e.g. during cleanings, irrigations, or aggressive periodontal procedures, such as root planning & scaling). The composition of the invention may be provided in any of the products defined herein.

Some embodiments of the present invention provides methods of whitening a tooth, wherein the tooth-whitening composition is applied to a tooth within five minutes of the first and second components being combined. In some embodiments, the tooth-whitening composition is applied to a tooth within three minutes of the first and second components being combined. In some embodiments, the tooth-whitening composition is applied to a tooth within two minutes of the first and second components being combined. In some embodiments, the tooth-whitening composition is applied to a tooth within one minute of the first and second components being combined.

In some embodiments, the tooth-whitening composition is applied to a tooth within thirty seconds of the first and second components being combined. In some embodiments, the tooth-whitening composition is applied to a tooth within fifteen seconds of the first and second components being combined.

In some embodiments, the viscosity of the first component is less than the viscosity of the second component. In some embodiments, the viscosity of the first component is the same as the viscosity of the second component.

Either chamber, compartment or component may contain additional ingredients which are typical to most oral care or mouthrinse formulations.

### EXAMPLES

### Example 1

Six concentrated mouthwash liquids were prepared as shown in Table 1. TSPP is the tetrasodium salt of pyrophosphoric acid. SAPP is the disodium salt of Pyrophosphoric acid. Poloxomer is a common nonionic surfactant from BASF. By varying the ratio of TSPP/SAPP, the pH of each formula is adjusted.

TABLE 1
Concentrated Mouthrinse Formulations

| | Formula Identification Number | | | | | |
|---|---|---|---|---|---|---|
| | 1 | 2 | 3 | 4 | 5 | 6 |
| Ingredient | Wt % | | | | | |
| Water | 77 | 77 | 77 | 77 | 77 | 77 |
| Glycerin | 20 | 20 | 20 | 20 | 20 | 20 |
| Poloxomer 407 | 2 | 2 | 2 | 2 | 2 | 2 |
| TSPP | — | 0.05 | 0.25 | 0.45 | 0.65 | 0.85 |
| SAPP | 0.9 | 0.85 | 0.65 | 0.45 | 0.25 | 0.05 |
| Peppermint Oil | 0.1 | 0.1 | 0.1 | 0.1 | 0.1 | 0.1 |

A stock solution of Lissamine Green (CI44090) was prepared in deionized water at a concentration of 1.73 mM. A stock solution of H₂O₂ was prepared in deionized water at a concentration of 1.4% (w/w) from commercial-grade H₂O₂. The series spectrophotometric measurements described below were collected using a Perkin Elmer Lambda 25 UV/Vis Spectrometer.

UV/VIS Calibration:

The following procedure is performed for each mouthwash liquid in Table 1. A first solution of 1.5 mL mouthwash liquid combined with 1.5 mL DI water is prepared and used to collect a background signal. A second solution of 1.5 mL mouthwash liquid combined with 1.5 mL water and 20 uL Lissamine Green B solution is prepared as a test sample. A spectral scan of the test sample from 400 nm-800 nm is collected at a scan speed of 480 nm/min with slit width of 1.0 nm. The spectrum of Lissamine Green is used to identify the maximum absorbance wavelength. After identifying the maximum absorbance at each pH, seven calibration samples are prepared containing varying amounts of Lissamine Green, as detailed in Table 2. The absorbance at each concentration is measured and used to generate a calibration curve relating Lissamine Green concentration to raw absorbance. Table 3 lists the calibration coefficient of Lissamine Green in each mouthwash liquid. The y-intercept was forced through 0. In each sample, the R² value is measured as R²>0.99, indicating a very good linear fit to experimental data.

TABLE 2
Calibration Samples for Lissamine Green (LG)

| Added LGB | Total Sample Volume | μM |
|---|---|---|
| (uL) | (mL) | LG |
| 0 | 3 | 0.00 |
| 5 | 3 | 2.88 |
| 10 | 3 | 5.77 |
| 15 | 3 | 8.65 |
| 20 | 3 | 11.53 |
| 25 | 3 | 14.42 |
| 30 | 3 | 17.30 |

TABLE 3
Calibration Constants for Lissamine Green (LG)

| Formula # | Constant (A/μM LG) | R\hat{ }2 |
|---|---|---|
| 1 | 0.0846 | 0.999 |
| 2 | 0.0836 | 0.999 |
| 3 | 0.0874 | 0.994 |
| 4 | 0.0795 | 0.999 |
| 5 | 0.656 | 0.998 |
| 6 | 0.666 | 0.999 |

Time Resolved Bleaching of Lissamine Green:

The following procedure is performed for each mouthwash liquid in Table 1. 1.5 mL mouthwash liquid is combined with 20 μL Lissamine Green solution and 1.5 mL H₂O₂ solution in a cuvette. The sample is placed immediately into the spectrometer, which has already been background-corrected. The typical elapsed time between reagent mixing and the first data point is 3-5 seconds. The absorbance of Lissamine Green at the maximum absorbance wavelength is monitored over a 1 minute period with a collection interval of 0.1 seconds. The absorbance is seen to reduce over time, or quench, as H₂O₂ oxidizes or reduces part of the conjugated pi system in the LG molecule. The raw absorbance values are convened to Lissamine Green concentration using the calibration coefficients in Table 3. Applying some simple theory, it is possible to determine the pseudo first-order rate constant governing the pH dependent bleaching of Lissamine Green. Although the pH-dependent rate constants are determined using LG instead of biologically relevant colored molecules, the data indicates that the activity of H₂O₂ as a bleaching substrate is strongly dependent on solution pH. This trend implicates a pH dependence of the reaction mechanism, which should remain true regardless of the exact structure of the colored molecule being bleached.

Theory:

The general rate equation for the bleaching of Lissamine Green (LG) is given as

$$Rate = - \frac{d [ L G ]}{d t} = {{k [ {H}_{2} {O}_{2} ]}^{a} [ L G ]}^{b}$$

a and b are the reaction orders with respect to H₂O₂ and Lissamine Green, respectively. In the limit where the concentration of H₂O₂ is much greater than the concentration of LG, we can assume that the concentration of H₂O₂ is essentially constant throughout the reaction. Simple examination of the relative concentrations of LG and H₂O₂ used in the current experiment verify that [H2O2]>>>[LGB]. This approximation allows a pseudo rate constant, k′, to be defined as

k′=k[H₂O₂]^{α}

The rate equation is thus simplified to

$$- \frac{d [ L G ]}{d t} = {{k}^{′} [ L G ]}^{b}$$

In the case where b=1, the reaction is described as pseudo first order integrated rate law becomes

ln [LG]=ln [LG]₀−k′t

This can be written alternatively as

[LG]=[LG]₀e^{-k′t}

It is apparent that a graph of ln [LG] vs. t would show a straight line with slope of −k′.

pH-Dependence of Rate Constants:

Lissamine Green bleaching in Mouthwash Liquid 5 is evaluated, where LG concentration (μM) is plotted against time (min). The apparent concentration of LG decreases from ˜10.5 μM to −0.0 μM over the 1 minute period. The starting concentration of LG is ideally 11.53 μM, indicating that some quenching has occurred prior to the t=0 experimental time point. A linear relation between ln [LG] and time is observed for all test solutions, indicating first order kinetics. The pseudo first order rate constants (k′) can be easily determined. Table 4 tabulates the pseudo first order rate constants for each test solution.

TABLE 4
Rate constants of LG bleaching

| Formula | pH | k′ (min⁻¹) |
|---|---|---|
| 1 | 4.5 | 3.83E−03 |
| 2 | 5.4 | 3.72E−02 |
| 3 | 6.2 | 1.70E−01 |
| 4 | 6.8 | 5.61E−01 |
| 5 | 8 | 4.70E+00 |
| 6 | 9.6 | 7.08E+00 |

TABLE 5

| | H2O2 level after 1 |
|---|---|
| Samples | week at 25 C. |
| 2% H2O2 buffered at pH 5 | 2.02% |
| 2% H2O2 buffered at pH 8 | 1.94% |

The data suggested that after 1 wk at RT, there is more peroxide loss at pH 8.

### Example 2

The whitening efficacy of an exemplary composition of the present invention and a comparative composition are compared.

Specifically, in-vitro stain removal (whitening) efficacy on HAP disk of @pH 5 vs. pH 8 as compared to de-ionized water (negative control) as compared to stained disk (Baseline). (Higher ΔE=greater efficacy).

TABLE 6

| Samples | Δ E |
|---|---|
| Deionized Water | 2.4 |
| 2% H2O2 buffered at pH 5 | 10.6 |
| 2% H2O2 buffered at pH 8 | 12.51 |

The data described in Table 6 (above) demonstrates that the compositions of the present invention deliver an unexpectedly increased level of whitening versus a similarly formulated peroxide containing composition which was not maintained at the pH of the compositions of the present invention. This data is even more unexpected in view of the results described in Table 5, wherein more peroxide is lost as pH increases.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the scope of the invention should be construed broadly as set forth in the appended claims.

1. A dual component oral care system comprising
a first component comprising a peroxygen compound having a first pH and
a second component comprising at least one salt of a weak mono or polyprotic acid
having a second pH
wherein the second pH is higher than the first pH and is less than 10.0;
wherein when combined the first and second components form a tooth-whitening composition having a pH greater than 6.0 and less than 10.0.

2. The oral care system of claim 1 wherein the second component comprises a salt of pyrophosphoric acid.

3. The oral care system of any preceding claim wherein the second component includes tetrasodium pyrophosphate.

4. The oral care system of any preceding claim wherein the second component includes disodium pyrophosphate.

5. The oral care system of any preceding claim wherein the second component comprises a mixture of two or more salts of pyrophosphoric acid.

6. The oral care system of claim 5 wherein the second component comprises tetrasodium pyrophosphate and disodium pyrophosphate.

7. The oral care system of claim 6 wherein the second component comprises tetrasodium pyrophosphate and disodium pyrophosphate in a ratio of from 20:1 to 1:20.

8. The oral care system of claim 7 wherein the second component comprises tetrasodium pyrophosphate and disodium pyrophosphate in a ratio of from 17:1 to 1:17.

9. The oral care system of any preceding claim wherein when combined the first and second components form a tooth-whitening composition having a pH of from 6.8 to less than 9.0.

10. The oral care system of any preceding claim wherein when combined the first and second components form a tooth-whitening composition having a pH of from 6.8 to 8.5.

11. The oral care system of any preceding claim wherein when combined the first and second components form a tooth-whitening composition having a pH of 7.5 to 8.5.

12. The oral care system of any preceding claim wherein when combined the first and second components form a tooth-whitening composition having a pH of about 8.0.

13. The oral care system of any preceding claim wherein the first component comprises less than 1% transition metal ions, metal oxides (e.g. titanium dioxide or magnesium dioxide).

14. The oral care system of any preceding claim wherein the first component comprises less than 0.1% transition metal ions, metal oxides (e.g. titanium dioxide or magnesium dioxide).

15. The oral care system of any preceding claim wherein the first component is substantially free of transition metal ions, metal oxides (e.g. titanium dioxide or magnesium dioxide).

16. The oral care system of any preceding claim wherein the pH of the first component is less than or equal to 7.0.

17. The oral care system of any preceding claim wherein the pH of the first component is from 1.0 to 7.0.

18. The oral care system of any preceding claim wherein the pH of the first component is from 4.0 to 7.0.

19. The oral care system of any preceding claim wherein the pH of the first component is from 4.0 to 6.8.

20. The oral care system of any preceding claim wherein the pH of the first component is from 4.5 to 5.5.

21. The oral care system of any preceding claim wherein the pH of the first component is from 4.8 to 5.2.

22. The oral care system of any preceding claim wherein the pH of the first component is about 5.0.

23. The oral care system of any preceding claim wherein the pH of the second component is from 7.1 to less than 9.0.

24. The oral care system of any preceding claim wherein the pH of the second component is from 7.5 to less than 9.0.

25. The oral care system of any preceding claim wherein the pH of the second component is from 7.5 to 8.5.

26. The oral care system of any preceding claim wherein the pH of the second component is about 8.0.

27. The oral care system of any preceding claim wherein the peroxygen compound is selected from one or more of peroxides, perborates, percarbonates, persulfates, perphohosphates, persilicates, peroxyacids and combinations thereof.

28. The oral care system of any preceding claim wherein the peroxygen compound is a peroxide.

29. The oral care system of any preceding claim wherein the peroxygen compound is hydrogen peroxide.

30. The oral care system of any preceding claim wherein the peroxygen compound is present in an amount of 0.01 to 20 weight % based on the total weight of the tooth whitening composition.

31. The oral care system of any preceding claim wherein the peroxygen compound is present in an amount of 0.01 to 10 weight % based on the total weight of the tooth whitening composition.

32. The oral care system of any preceding claim wherein the peroxygen compound is present in an amount of 0.01 to 7.5 weight % based on the total weight of the tooth whitening composition.

33. The oral care system of any preceding claim wherein the peroxygen compound is hydrogen peroxide present in an amount of 0.01 to 3 weight % based on the total weight of the tooth whitening composition.

34. The oral care system of any preceding claim wherein the tooth whitening composition is a mouthwash.

35. The oral care system of any preceding claim wherein
the first pH is acidic,
the second pH is alkaline and
the pH of the combination of the first and second components is alkaline
wherein the second component acts as a buffer and the combination of the first and second components has a pH that is less than or equal to the pH of the second component.

36. The oral care system of any preceding claim wherein the viscosity of the first component is less than the viscosity of the second component.

37. The oral care system of any preceding claim wherein the viscosity of the first component is the same as the viscosity of the second component.

38. A method of tooth whitening comprising combining
a first component comprising a peroxygen compound and having a first pH and
a second component comprising at least one salt of a weak mono or polyprotic acid and having a second pH
wherein the second pH is higher than the first pH and is less than 10.0;
to form a tooth-whitening composition having a pH of greater than 6.0 and less than 10.0 and applying the tooth-whitening composition to a tooth.

39. The method of claim 38, wherein the tooth-whitening composition is applied to a tooth within five minutes of the first and second components being combined.

40. The method of claim 38, wherein the tooth-whitening composition is applied to a tooth within three minutes of the first and second components being combined.

41. The method of claim 38, wherein the tooth-whitening composition is applied to a tooth within two minutes of the first and second components being combined.

42. The method of claim 38, wherein the tooth-whitening composition is applied to a tooth within one minute of the first and second components being combined.

43. The method of claim 38, wherein the tooth-whitening composition is applied to a tooth within thirty seconds of the first and second components being combined.

44. The method of claim 38, wherein the tooth-whitening composition is applied to a tooth within fifteen seconds of the first and second components being combined.

45. Use of a dual component oral care system according to any of claims 1 to 37 for whitening a tooth.
